(19) United States
(12) Patent Application Publication
(10) Pub. No.: US 2016/0331989 A1
(43) Pub. Date: Nov. 17, 2016

(54) Plasma Pad

(71) Applicant: Kwangwoon University Industry-Academic Collaboration Foundation, Seoul (KR)

(72) Inventors: Guangsup Cho, Seoul (KR); Yunjung Kim, Seoul (KR)

(73) Assignee: Kwangwoon University Industry-Academic Collaboration Foundation, Seoul (KR)

(21) Appl. No.: 15/002,399

(22) Filed: Jan. 21, 2016

(30) Foreign Application Priority Data
May 14, 2015 (KR) 10-2015-0067004

(51) Int. Cl. A61N 1/44; A61N 1/04

(52) U.S. Cl. CPC A61N 1/44; A61N 1/0484

(57) ABSTRACT

The present invention provides a plasma source in the form of a pad so that a user discretionally wears the plasma pad, i.e., present invention provides a wearable plasma pad that is conveniently worn by a user for the purpose of a long period of contact to plasma, fabricated discretionally into various shapes and areas and appropriately responds to the body part that requires a plasma therapy. The plasma pad according to the present invention is fabricated with a flexible base plate on which a high voltage electrode coated or layer with dielectric materials and a ground electrode are arrayed. AC voltage is applied to the high voltage electrode so that the plasma pad generates plasma between the electrodes by means of atmospheric discharge. Products such as patch, bandage, cap, hair-band, socks, etc. may be achieved based on the plasma pad thus fabricated.

### CROSS-REFERENCE TO PRIOR APPLICATIONS

This application claims priority of Korean Patent Application No. 10-2015-0067004, filed on May 14, 2015, which is hereby incorporated by reference in its entirety.

### TECHNICAL FIELD

The present invention relates to an apparatus to generate atmospheric surface plasma used for skin aesthetics, skin disorder treatment, hemostasis and injury treatment consequent upon skin damage, hair growth promotion on the scalp, etc. and, more specifically, to an apparatus to generate atmospheric surface plasma, based on a plasma pad that generates plasma on a predetermined surface, that assumes various shapes such as plasma patch, plasma bandage, plasma sock, plasma cap, plasma hairband and the like for the purpose of skin aesthetics and skin disorder treatment, etc.

### BACKGROUND ART

Plasma at atmospheric pressure, or atmospheric plasma, is a kind of plasma generated by atmospheric discharges via which particles of radical species are generated such as reactive nitrogen and oxygen. Such radical species react to the surface of skin, etc. bringing about effects including disinfection, activation of skin tissue, skin cell regeneration, hemostasis with blood coagulation, promotion of hair growth, etc., on the basis of which plasma may be exploited in skin aesthetics and treatment of various skin disorders and skin injuries. More specifically, plasma may be used in the field of skin aesthetics for removing wrinkles, recovering skin elasticity, etc. via anti-aging and activating face or body skin and in the field of skin disorder treatment, which accounts for treating chronic skin diseases, etc. such as tinea pedis by removing various bacteria in the skin. Plasma may be used also in disinfecting the affected area, hemostasis, recovering the affected area, treating and recovering burns, preventing alopecia, promoting hair growth, etc.

Plasma sources are developed and fabricated for the therapeutic or aesthetic purposes in order to obtain such advantageous effects of plasma.

Plasma at atmospheric pressure is generated with ease in the dielectric barrier discharge (DBD) mode by means of a high AC voltage. Existing plasma treatment devices for aesthetic and medical purposes have been developed into plasma stamps, sticks, rollers, combs, etc. that apply plasma onto the body to treat. They are, however, difficult to handle when the affected area is required to be exposed to plasma for a long period of time that ranges from a few dozen minutes or a few hours or if a significantly large area of skin is to contact to plasma.

Korea Patent Registration No. 10-1292268 refers also to a plasma-based treatment device but a user may not facilitate using the technology by him or herself in that the device should be secured at a structure while the affected area should be exposed to plasma.

### DISCLOSURE OF INVENTION

### Technical Problem

Accordingly, the present invention provides a plasma-based treatment device that affords convenience in use for aesthetic or medical purposes when a long period of treatment time or a considerably large area to expose to plasma for treatment is required.

### Technical Solution

To achieve the objectives, the present invention provides a plasma source fabricated into a pad so that a user discretionally wears the plasma pad according to the application.

More specifically, the present invention provides a plasma pad that is worn by a user, which facilitates easy contact to plasma for a long period and, further to that, a wearable plasma pad that is fabricated discretionally in terms of the geometry and area so that the plasma is applied, as necessary, to the body part.

The present invention provides a plasma pad that generates plasma on a predetermined surface as well as is flexible and wearable.

A plasma pad provided by the present invention is made by arraying electrode(s) on the surface of a flexible or stretchable base. The electrode set is basically a high voltage electrode and a ground electrode may be added as necessary. The high voltage electrode is constituted by enclosing a metallic electrode with dielectric layer. The metallic electrode in the high voltage electrode comprises metallic surfaces or lines. The geometry of the high voltage electrode may be varied in many ways. The dielectric layer in the high voltage electrode may comprise a wire covered with a dielectric layer with a high dielectric strength or be fabricated with a polymer film with a high dielectric strength on which a metallic pattern is formed to have a predetermined geometry. The ground electrode may comprise a covered with dielectrics or bare metallic wire or be fabricated with a polymer film on which a metallic pattern is formed.

Such a plasma pad provided by the present invention may be fabricated into various shapes to add convenience in use. More specifically, the present invention may provide a plasma patch that wraps an adhesive film around the edge of a basic plasma pad, a plasma bandage to which a basic plasma pad is extended to form a bandage to band a wrist or ankle, a plasma sock for which a basic plasma pad is mounted on the bottom surface of a sock, a plasma cap for which a basic plasma pad is mounted on the internal surface of a hairband, etc.

The present invention applies the plasma pad to a patch, band, bandage, sock or cap in order to afford therapeutic convenience in respect to the body part of interest and add therapeutic synergy because the plasma pad applications are used in parallel with each ointment and/or preparation as a due therapy.

Meanwhile, the present invention makes a user control the power system of the plasma apparatus, and accordingly the amount or period of the plasma generation, in order to prevent an electrical shock or thermal damage, which accounts for the mode to secure the electrical safety of the plasma pad. The power system may be a typical domestic power supply (AC 60 Hz, 110/200 V) or a wireless one that adopts a charging/discharging technique by means of a secondary battery.

The plasma pad comprises a plasma source that is a DBD in order to micro-discharge the atmosphere without additional gas supply.

A high voltage that is an AC voltage the frequency of which ranges from a few Hz to a few dozen kHz is to be applied from the power system to the high voltage electrode to generate plasma according to the present invention. The high voltage may have various modulated waveform including sine and pulse. The voltage for the AC power ranges from a few hundred V to a few kV while a DC-AC inverter is typically for that purpose.

A wire with a high electrical conductivity such as a copper wire is used for the high voltage electrode while a plurality of strands of such wire may be used to guarantee the flexibility.

The dielectric materials to cover the high voltage wire are selected from silicons, synthetic rubbers with a high dielectric strength, polymers, etc. to achieve flexibility.

The film on which the high voltage electrode is patterned is selected from heat-resistant, electrically insulating and flame-resistant polymers while a polyimide (PI) film employed for a flexible printed circuit board (FPCB) is preferred among other various polymers including polystyrene, polyethylene (PE), polypropylene (PP), and polycarbonate.

### Advantageous Effects of Invention

A plasma pad according to the present invention contacts to the skin activating the skin tissue and disinfecting the skin to keep the skin hygienic to bring about therapeutic effects. To create the therapeutic synergy the plasma pad may be used in parallel with medical preparations such as ointment so that the plasma facilitates the preparation permeation into the skin.

The plasma pad according to the present invention is adhesive, which is convenient and effective in procedures in the field of skin aesthetics and dermatology involved with a long period of treatment time and/or a large area of the affected part.

The plasma pad according to the present invention provides a dedicated power system equipped with a DC-AC inverter and, accordingly, may be operated via a typical domestic power outlet. The plasma pad may be, however, operated by means of additional DC or rechargeable cell(s), which affords the plasma pad portability and therapeutic convenience irrespective of locations.

In addition, the plasma pad according to the present invention is powered by a high voltage with a trace amount of current, which guarantees the electrical safety, without electrical shocks, of the apparatus as well as controls the plasma temperature within a safe range.

### BRIEF DESCRIPTION OF DRAWINGS

FIGS. 1A and 1B show a group of perspective views to illustrate the configuration of a plasma pad according to an embodiment of the present invention which generates plasma at the intersections between two wires. FIG. 1A is a disassembled perspective view of a plasma pad which generates plasma in the proximity of the intersections between a high voltage electrode comprising a covered wire and a ground electrode comprising a covered or bare wire. FIG. 1B is a perspective view that illustrates electrodes of a plasma pad which generates plasma between a high voltage electrode 200 comprising a covered wire and a ground electrode 300 comprising a bare wire arrayed alongside with each other.

FIG. 2A is a perspective view of an electrode section of a plasma pad where a high voltage electrode is arrayed on a bottom polymer film and an upper polymer film is laid on the high voltage electrode then a ground electrode comprising a covered or bare wire is arrayed in a direction to intersect the high voltage electrode on the upper polymer film.

FIG. 2B is a disassembled perspective view of a plasma pad where the high voltage electrode lines are fabricated on the bottom polymer film and the ground electrode lines are fabricated on the upper polymer film, respectively, then arrayed in a parallel direction to each other so that the assembly generates plasma between the two electrodes.

FIG. 2C is a disassembled perspective view of a plasma pad where the high voltage electrode like bids lines are fabricated on the bottom polymer film and the ground electrode ring-like lines are fabricated on the upper polymer film, respectively, then bids arrayed inside of ring respectively so that the assembly generates plasma between the two electrodes.

FIGS. 3A and 3B show an entire configuration of a system that connects a plasma pad to a power system. FIG. 3A illustrates a connection system that wires a plasma pad and a power system that is to be connected to a typical domestic power outlet. FIG. 3B illustrates a power system, including a charging/discharging device, in the wireless mode using a secondary battery.

FIGS. 4 through 8 are product exemplifications to which a plasma pad according to the present invention is applied.

FIG. 9 illustrates an actual plasma pad that has been achieved according to the present invention and is generating plasma.

### BEST MODE

Preferred embodiments according to the present invention are now described below with reference to accompanying drawings.

A plasma pad according the present invention comprises a flexible base plate 100, a high voltage electrode 200 or ground electrode 300 arrayed on the base plate 100 and a gauze cover 500 that covers the electrodes 200, 300. The base plate 100 and the gauze cover 500 are preferably selected from fiber or synthetic fiber fabrics and may be made of nonwoven, thin leather, silicon, polymer, etc. The base plate 100 and gauze cover 500 are preferably made of air permeable materials while a plurality of perforations are additionally hollowed to facilitate ventilation.

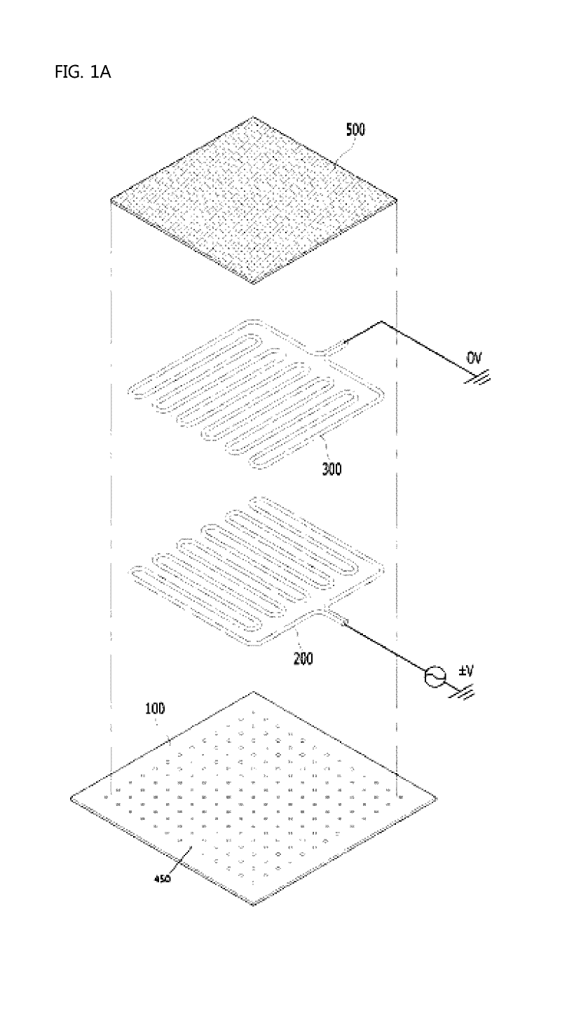

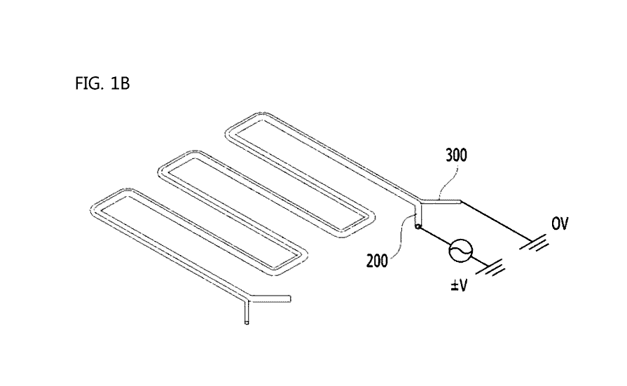

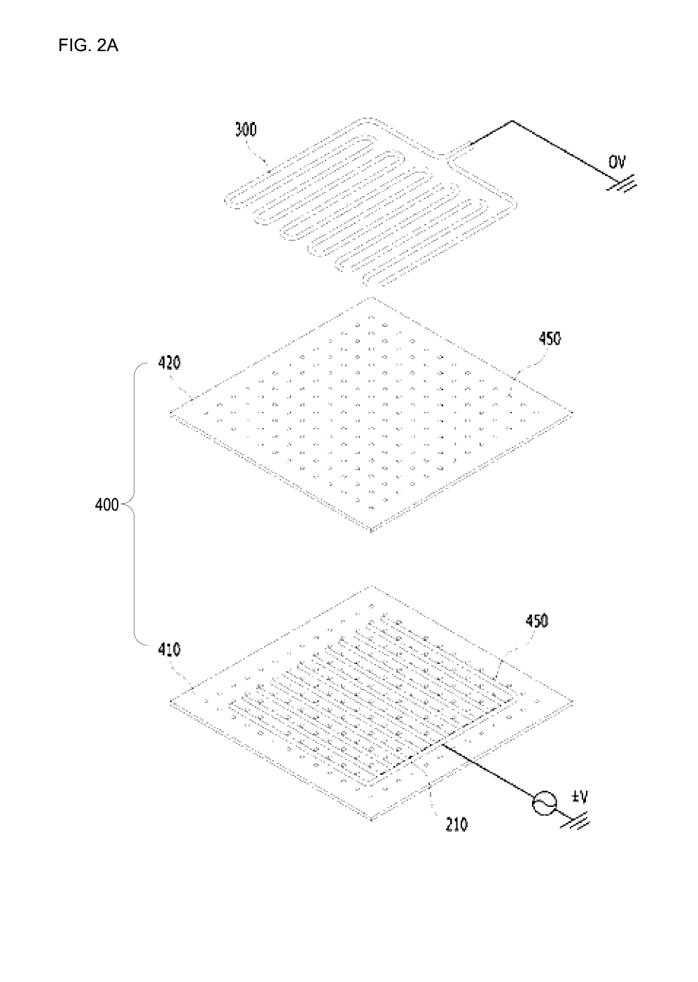

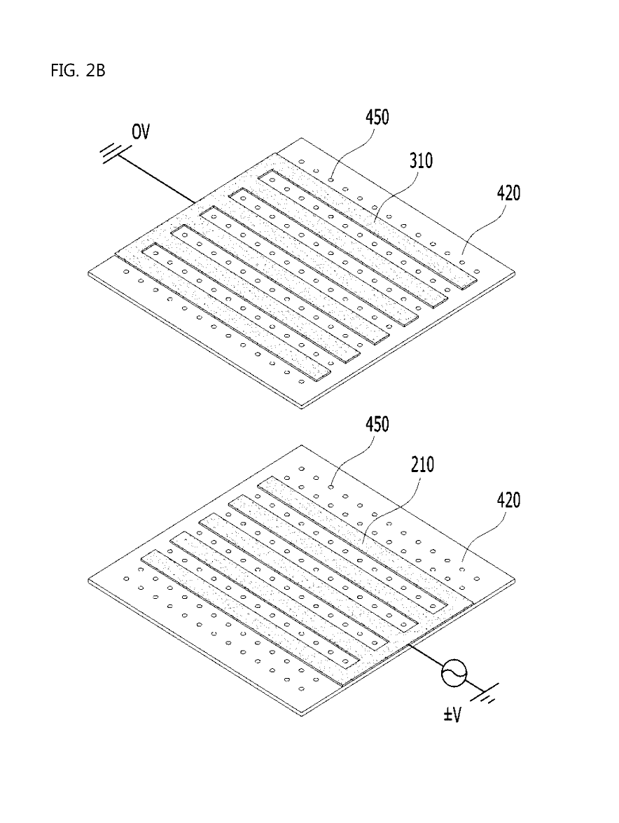

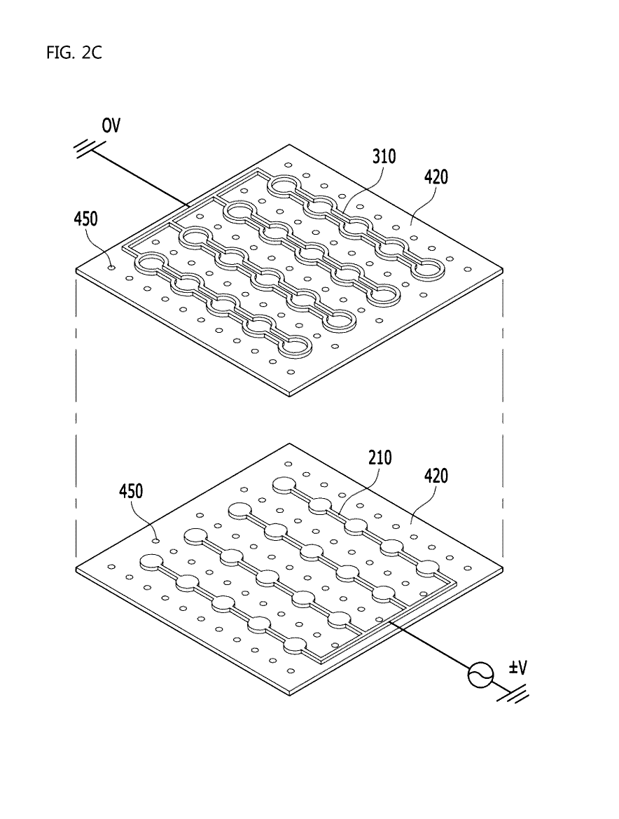

Electrodes of the plasma pad according to embodiments of the present invention comprise covered or bare conductive wires or are formed on a polymer film. Such a plasma pad generates plasma at the intersections between two electrodes as illustrated in FIG. 1 or between two electrodes arrayed alongside with each other as illustrated in FIG. 2.

The high voltage electrode 200 may comprise a dielectrics-covered conductive wire while the ground electrode 300 may a covered or typical, bare conductive wire. The covered conductive wire may be required to have a diameter of 2 mm or less to minimize the thickness of the plasma pad. The bare conductive wire for the ground electrode may also have a diameter of 2 mm or less or be fabricated with a plurality of strands the diameter of which is 1 mm or less.

A PI film with a thickness ranging from a few dozen to a few hundred μm is preferably selected for the polymer film 400 on which the high voltage electrode 200 or the ground electrode 300 is configured where the electrodes are, in general, formed in a printing mode by means of conductive paste.

FIG. 1 is a group of perspective views to illustrate the configuration of a plasma pad according to an embodiment of the present invention which generates plasma at the intersections between two wires. FIG. 1A is a disassembled perspective view of a plasma pad which generates plasma in the proximity of the intersections between a high voltage electrode comprising a covered wire and a ground electrode comprising a covered or bare wire. FIG. 1B is a perspective view that illustrates electrodes of a plasma pad which generates plasma between a high voltage electrode 200 comprising a covered wire and a ground electrode 300 comprising a bare wire arrayed alongside with each other.

FIG. 1A illustrates a configuration of a plasma pad wherein the high voltage electrode 200 as a covered conductive wire is arrayed in a constant direction on the base plate 100 with perforations 450, and at a right angle to, which the ground electrode 300 as a covered or bare conductive wire is arrayed. Plasma is generated in the DBD mode in the proximity of the intersections between the latitudinally or laterally arrayed high voltage electrode 200 and the longitudinally arrayed ground electrode 300 and the amount of the plasma generation is controlled on the plasma pad surface by adjusting the gap between the high voltage electrode and the ground electrode thus arrayed.

Eventually, the gauze cover 500 may be required to cover the plasma pad thus fabricated in order to create a sense similar to that of conventional gauze when the plasma contacts to the skin.

FIG. 1B is a perspective view that illustrates only the electrode section.

FIG. 1B illustrates electrodes arrays, one 200 of the two electrodes made of a conductive wire covered with dielectric materials on which voltage ±V is applied and the other 300 made of a conductive wire bared or covered with dielectric materials which is grounded. Plasma in generated between the two electrodes that are proximate to each other.

FIG. 2A is a disassembled perspective view that illustrates a configuration of a plasma pad wherein a high voltage electrode 210 is formed on the polymer film 410 and the ground electrode 300 of a typical conductive wire is arrayed on an upper polymer film 420 which is laid on the high voltage electrode 210 that is formed on a bottom polymer film 410 laterally then the ground electrode 300 of a typical conductive wire either covered or bare is longitudinally arrayed thereon. A PI film with a thickness ranging from a few dozen to a few hundred μm is preferably selected for the polymer film 400 (including 410 and 420) on which the high voltage electrode 210 is configured where the electrode is, in general, formed in a printing mode by means of conductive paste. But the electrode forming mode is not limited to printing and may include, inter alia, vapor deposition.

Air permeable perforations 450 are hollowed at a constant interval in between through the polymer film 400 to secure good ventilation. The polymer film 400 on which the high voltage electrode 210 is formed attaches, as in an embodiment illustrated in FIG. 1A, onto the base plate 100 while the gauze cover 500 may be required to cover the electrode 300 from above. If the base plate 100 attached with the polymer film 400 is made of a non-air permeable material, perforations in the base plate 100 may be required to be bored at positions corresponding to those of the air permeable perforations 450 hollowed through the polymer film 400.

FIG. 2B is a disassembled perspective view of a plasma pad where a high voltage electrode 210 and a ground electrode 310 are fabricated on a polymer film, respectively, then arrayed in a parallel direction to each other so that the assembly generates plasma between the two electrodes with a constant gap in between.

FIG. 2B is a disassembled perspective view that illustrates a plasma pad that generates plasma between two conductive electrodes 210, 310 arrayed alongside with each other on the polymer film 420 where the high voltage electrode 210 is arrayed in a longitudinal direction on the bottom polymer film 420, the upper polymer film 420 covers the high voltage electrode 210 from above then the ground electrode 310 is arrayed on the upper polymer film 420. Each of the high voltage electrode 210 and the ground electrode 310 is alternatively engaged respectively with a constant gap. Such a configuration is preferred because a smaller gap between the electrodes brings about a smaller amount of discharge voltage. However, if the two electrodes superimpose on an area, the area may potentially lead to dielectric breakdown, which indicates a prohibited situation. Therefore, the two electrodes should run alongside with each other, not be superimposed onto each other. The air permeable perforations 450 are hollowed so that ventilation is secured through the two polymer films 420 when the two polymer films 420 stick to each other.

FIG. 2C is a disassembled perspective view of a plasma pad where a high voltage electrode 210 like bids line on a polymer film 420 and a ground electrode 310 like rings line are fabricated on a polymer film 420, respectively, then bids arrayed inside of ring respectively with a gap so that the assembly generates plasma between the two electrodes. Such a configuration is preferred because a smaller gap between the electrodes brings about a smaller amount of discharge voltage. The air permeable perforations 450 are hollowed so that ventilation is secured through the two polymer films 420 when the two polymer films 420 stick to each other.

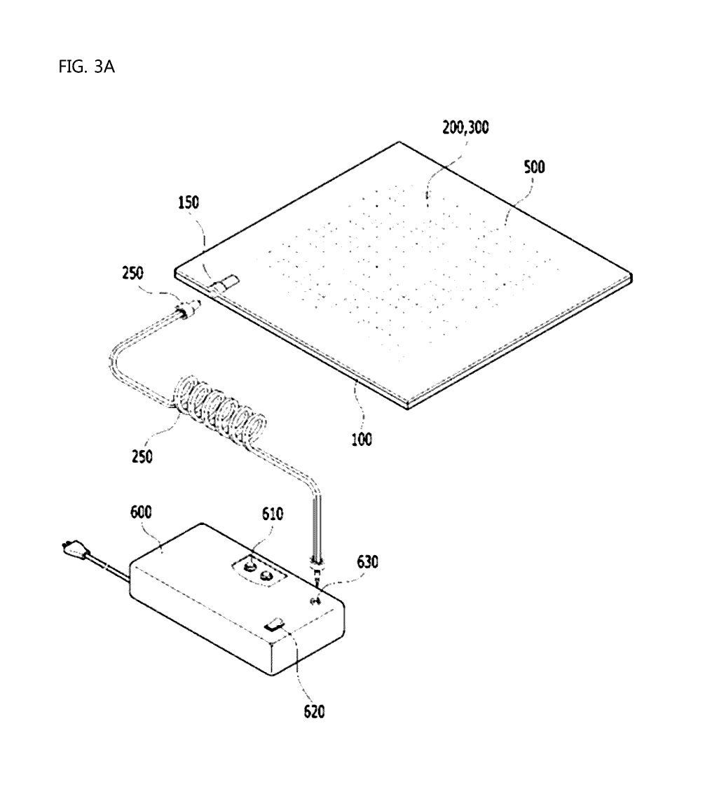

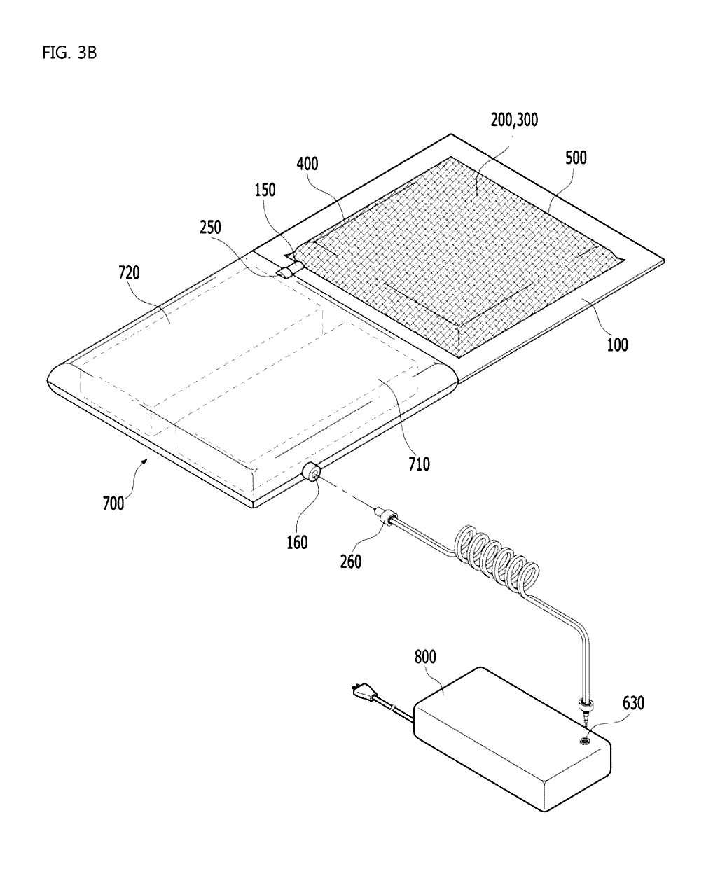

FIG. 3 illustrates a power system of the plasma pad. FIG. 3A is a power system for domestic use while FIG. 3B is a power system in a charging/discharging mode by means of a secondary battery.

FIG. 3A illustrates a configuration of a plasma pad that employs a separate electric power supply 600 and installs on a side of the base plate 100 a socket 150 to be connected to the electric power supply. The electric power supply 600 is equipped with a terminal connection 630. A cable 250 comprises a plug that is connected to the socket 150 at one end and a terminal that is connected to the terminal connection 630 at the other end. The cable 250 secures flexibility so that the cable 250 varies with ease the lineal distance. The socket has two power pins in the inside. The plug to be plugged into the socket 150 also has two power pins and assumes a cable comprising two conductive wires. The electric power supply basically uses a typical power source (AC 60 Hz, 110/220 V). An SMPS and a DC-AC inverter that convert typical power to DC power are embedded in the electric power supply 600. In addition, an adjusting terminal 610 and a switch 620 that control the amount and period of the plasma generation are installed to the electric power supply 600.

FIG. 3B illustrates an electric power system in a charging/discharging mode using a secondary battery. The electric power supply is configured into a wireless electric power supply that is wearable using batteries for the convenience of the user and may be reused with the secondary battery recharged by means of a recharging device. The electric power supply comprising secondary battery and an inverter is flat and may be worn being contained in a pocket 700 connected to the plasma pad. The secondary battery is flat, selected from lithium polymer batteries, lithium ion batteries, etc. and ranges from a few to a few dozen volts in terms of DC volt. The DC-AC inverter that employs as the power source a secondary battery may also be flat. A charger 800 is connected to a typical power source via a socket for charging a secondary battery.

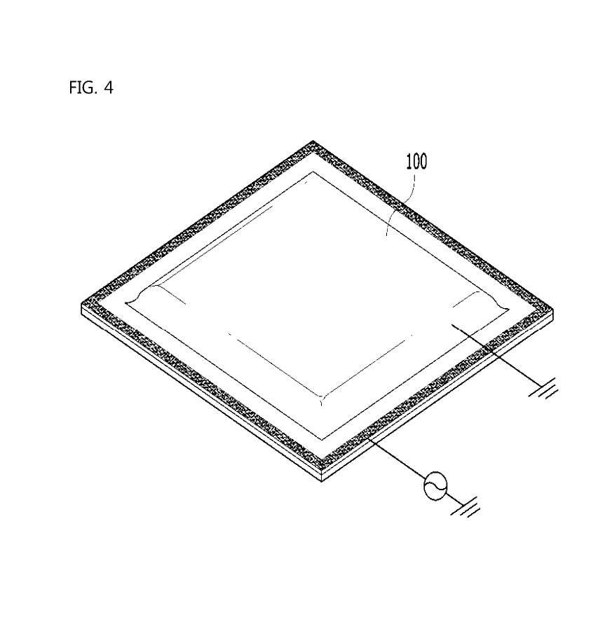

FIGS. 4 through 8 are product exemplifications to which a plasma pad according to the present invention is applied.

FIG. 4 illustrates a plasma patch for which an adhesive film wraps around the edge of the patch or the entire gauze so that the patch attaches to the skin.

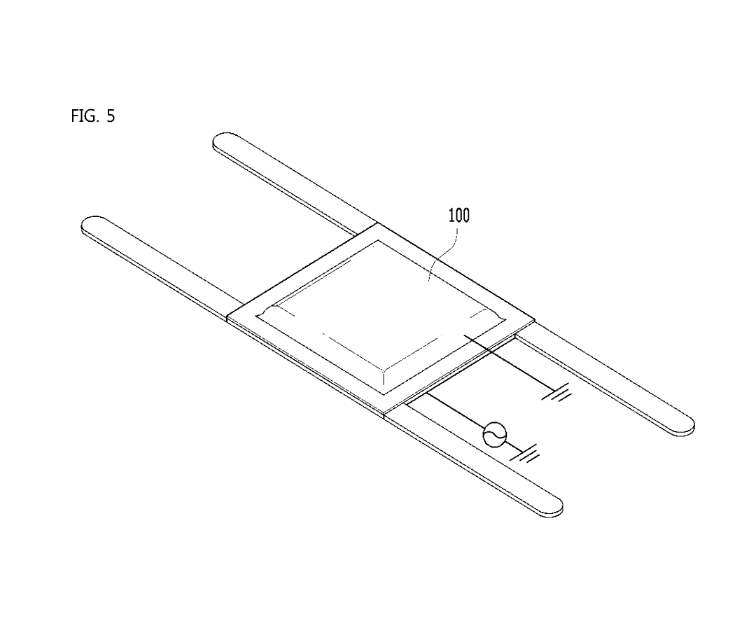

FIG. 5 illustrates a plasma bandage for which strips are mounted so that the bandage winds up and attaches to, and accordingly ties up, the wrist or ankle.

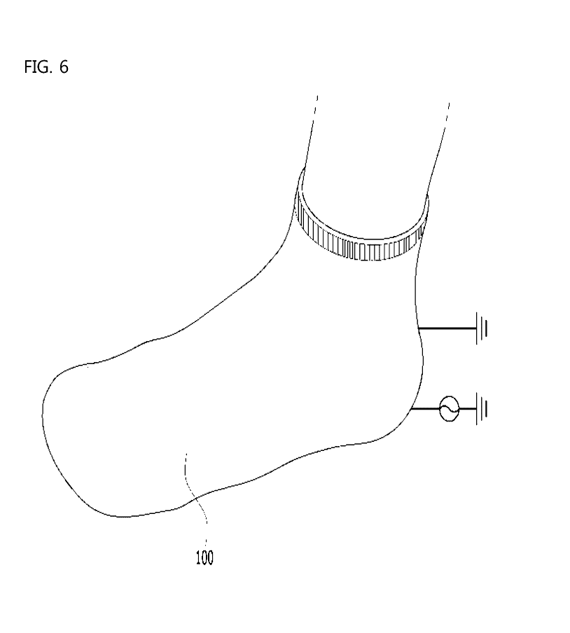

FIG. 6 illustrates a plasma sock inside which a plasma pad is installed on the bottom side.

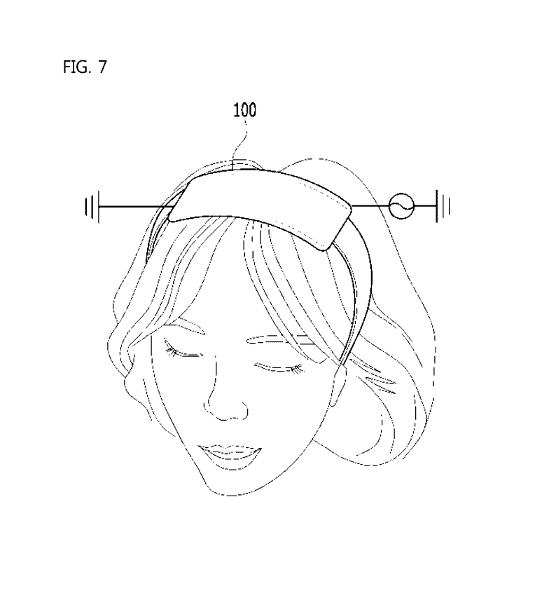

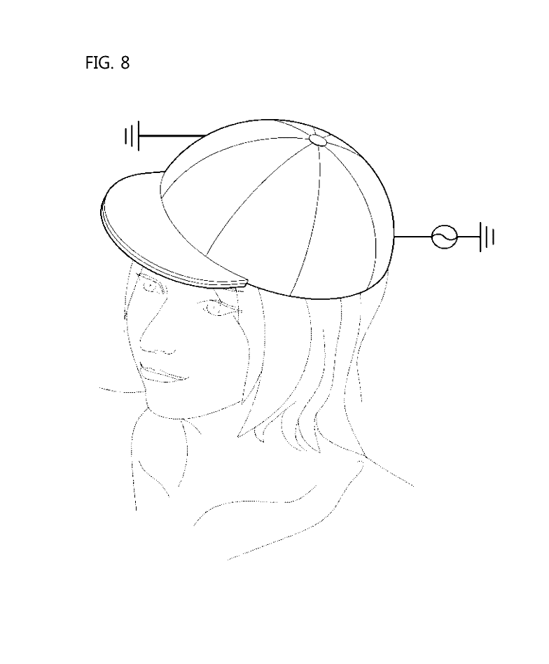

FIG. 7 and FIG. 8 illustrate a plasma hairband and plasma cap, respectively, with which a plasma pad is equipped.

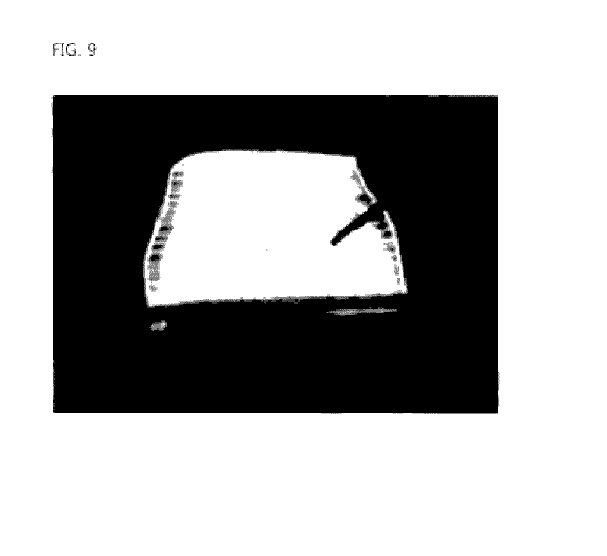

FIG. 9 illustrates an actual plasma pad that generates plasma according to the present invention with covered conductive wires arrayed as in FIG. 1A. As in FIG. 1A, the high voltage electrode 200 is arrayed on the gauze base plate 100 with a predetermined gap in between on which the ground electrode 300 is arrayed so that the ground electrode 300 intersects the high voltage electrode 200 at a right angle. An AC voltage of 1.5 kV at 60 kHz is applied to the covered conductive wire on the bottom while the covered conductive wire of the ground electrode 300 is arrayed in the latitudinal direction on the covered conductive wire of the bottom and grounded. The covered conductive wire has a diameter of 1 mm while the plasma pad has an area of 10 cm×15 cm. Plasma is generated at a voltage of about 1.5 kV with a current of about 7 mA.

The specific measurements referred to in the embodiments and experimental examples thus far are exemplary and subject to modification as necessary. It must be understood that a person skilled in the art can modify the present invention into specific variations without change in the technical thoughts and critical features. Therefore, the embodiments described thus far must be interpreted to be just exemplary, not to limit the present invention. The scope of the present invention is to be clarified by the Claims of the present invention, not by detailed description thus far while all the variations derived from the Claims of the present invention and the equivalents thereof must be interpreted to be included in the scope of the present invention.

### REFERENCE NUMERALS

100: Base plate
120: Ventilation hole
150, 160, 250, 260: Socket
200, 210: High voltage electrode
300, 310: Ground electrode
400: Polymer film
410: Bottom polymer film
420: Upper polymer film
450: Ventilation hole
500: Gauze-cover
600: Electric power supply
610: Adjusting terminal
620: Switch
630: Terminal connection
700: Pocket
710: Secondary battery
720: DC-AC inverter
800: Charger

What is claimed is:

1. A plasma pad, including: a base plate made of a flexible material; a first metallic electrode arrayed on the base plate; and a second metallic electrode arrayed on the first metallic electrode with dielectrics between the first metallic electrode and the second metallic electrode, wherein the plasma pad generates plasma for the purpose of skin therapies by discharge between the first metallic electrode and the second metallic electrode by connecting an AC power source between the first metallic electrode and the second electrode then applying AC voltage between the first metallic electrode and the second metallic electrode.

2. The plasma pad of claim 1, wherein geometry of the first electrode and the second electrode, respectively, includes linear, curved or circular sections and plasma is generated at the positions where the metallic electrode and the second electrode intersect at a right angle.

3. The plasma pad of claim 1, where the geometry of the first electrode and the second electrode, respectively, includes linear, curved or circular sections and plasma is generated between the first metallic electrode and the second electrode that are arrayed alongside with each other.

4. The plasma pad of claim 1, where voltage is applied to one of the first electrode and the second electrode to generate plasma and the other electrode is grounded.

5. The plasma pad of claims 1, where positive voltage is applied to one of the first metallic electrode and the second metallic electrode and negative voltage is applied to the other electrode.

6. The plasma pad of claim 1, where one electrode to which voltage is applied of the first electrode and the second electrode comprises a conductive wire covered with a dielectric material and the other electrode that is grounded comprises a covered or bare conductive wire.

7. The plasma pad of claim 1, where one electrode to which voltage is applied of the first electrode and the second electrode is formed on a polymer film and covered with an upper polymer film from above and the other one electrode as a grounded electrode comprising a conductive wire is arrayed on the upper polymer film.

8. The plasma film of claim 1, where all the first metallic electrode and the second metallic electrode are formed on a polymer film.

9. The plasma pad of claim 7, where the electrode formed on the polymer film is formed by printing conductive paste on the polymer film.

10. The plasma pad of claim 8, where the electrode formed on the polymer film is formed by printing conductive paste on the polymer film.

11. The plasma pad of claim 1, where the first metallic electrode and the second metallic electrode arrayed on the flexible base plate are covered a gauze cover.

12. The plasma pad of claim 1, where the electric power supply that applies AC voltage includes a DC-AC inverter, applies a voltage that ranges from one to ten kV and applies voltage that has a sine or modulated waveform with a frequency that ranges from one to a hundred kHz.

13. The plasma pad of claim 1, where the AC electric power supply uses as the power source of an inverter the DC power of a secondary battery, operates the plasma pad in a wireless mode and recharges the secondary battery.

14. A plasma patch that includes the plasma pad of claim 1 and attaches to the skin by being equipped with an adhesive material around the edge of the patch.

15. A plasma bandage that includes the plasma pad of claim 1 and attaches to the skin by being equipped with an adhesive material around the edge of the plasma bandage.

16. A plasma sock including the plasma pad of claim 1 on the bottom of the plasma sock.

17. A plasma cap including the plasma pad of claim 1 on the internal surface of the plasma cap.

18. A plasma hairband including the plasma pad of claim 1 on the internal surface of the plasma hairband.
